(19) United States
(12) Patent Application Publication
(10) Pub. No.: US 2009/0112182 A1
(43) Pub. Date: Apr. 30, 2009

(54) METHOD AND APPARATUS FOR VASCULAR ACCESS

(75) Inventor: Mehdi RAZAVI, Houston, TX (US)

Correspondence Address: CONLEY ROSE, P.C.;David A. Rose, P. O. BOX 3267, HOUSTON, TX, 77253-3267, US

(73) Assignee: TEXAS HEART INSTITUTE, Houston, TX (US)

(21) Appl. No.: 12/261,688

(22) Filed: Oct. 30, 2008

Related U.S. Application Data
(60) Provisional application No. 60/984,154, filed on Oct. 31, 2007.

(51) Int. Cl. A61M 29/02

(52) U.S. Cl. 604/507; 604/104

(57) ABSTRACT

A vascular access device comprises a bridge having first and second bores therethrough, a dilator in the first bore, and a needle guide in the second bore. The second bore is at an angle with respect to the first bore such that the needle guide is at the angle with respect to the dilator. The device is useful, for example to achieve dual site entrance to a blood vessel.

### CROSS-REFERENCE TO RELATED APPLICATION

This application claims priority to provisional patent application U.S. Ser. No. 60/984,154 filed Oct. 31, 2007, entitled “Method and Apparatus for Vascular Access,” and incorporated herein by reference.

### BACKGROUND

Vascular access is a necessary step in many medical procedures. Access to the vascular system is a required part for any situation in which temporary or permanent access to the vasculature is desired in the course of a medical procedure. Originally such access was commonly obtained by use of surgical techniques in which direct exposure of the target vessel was accomplished via blunt or fine surgical dissection. Subsequently, direct puncture of the vessel would enable its penetration and instrumentation.

This “cut down” technique was eventually replaced by percutaneous vascular techniques, the most common of which is the modified Seldinger technique. In this technique initial access is obtained via percutaneous puncture and direct access with a hollow bore needle. Return of blood (arterial or venous, depending on the vessel accessed) signals the needle's intravascular position. A wire is then advanced through the needle, into the vascular space. The needle is then withdrawn and the wire is left in place. Finally, a sheath with a dilator within it is advanced over the needle. The wire guides this apparatus into the vascular space. The wire and dilator are withdrawn, leaving the sheath within the vessel. This sheath then acts as a thoroughfare through which vascular access is immediately available.

In many circumstances multiple access sites for the same vessel is required. Such circumstances include, but are not limited to, the need for implantation of multiple pacing wires or leads or catheters for electrophysiology studies or cardiac ablation, and multiple indwelling catheters for chronic medication infusion. In such cases multiple access sites are obtained simply by repeating the aforementioned steps as many times as needed. This necessitates repeated episodes of achieving vascular access by penetration of a hollow bore vascular access cannula or needle. Each time access is achieved an individual wire is placed in the needle within the target vessel, the needle is removed, and a sheath with a dilator is advanced over it.

Very often and for a variety of reasons vascular access can be challenging. This can be due to, as an example, scarring or narrowing of vessels, obesity, or atypical anatomy. In such cases the need for multiple access sites can add even more complexity and risk to any attempts at vascular access. Examples of such complications include penetration of adjacent tissue, which in turn can lead to vascular complications such a bleeding, blood clots, aneurysm formation, or fistula formation.

Unfortunately in a situation wherein multiple access sites are required, obtaining the first access site does not convey straightforward subsequent access of the same vessel. Common techniques utilized to repeatedly access the same vessel after an initial successful access and “wiring” of the vessel include angling the needle parallel to the access of the wire, using fluoroscopy and the accessed guide wire as roadmaps for the hollow bore cannula or needle, or placing multiple wires in the same sheath. The first two of these are unreliable because tracking of the wire by the needle can be limited by the inability to have a three dimensional understanding of the relative positioning of the needle relative to the wire since fluoroscopic imaging is performed in two dimensions. Very often the needle will appear to be in contact with the wire and yet vascular access is not possible.

In the case of the multiple wire technique, after the first and only wire has accessed the vessel, a sheath and dilator is advanced over it per the technique described above. Then multiple wires are inserted through the lumen of the sheath. These wires enter the vessel through the sheath. The sheath is then removed, the wires retained in the vessel, and multiple individual sheaths with dilators within them are advanced over individual wires. However, in this case all access sites have been obtained through the same perforation (the original and only percutaneous cannulation of the vessel through which the original wire was placed). Thus multiple access sites are advancing through a single penetration site, both at the skin and vessel levels. Such immediate contact creates many technical limitations including but not limited to collapse of sheaths due to direct contact with other immediate sheaths and catheters, inability to effectively manipulate adjacent catheters or leads because of such contact, inability to suture leads, and the like. Additionally, because there is physically a single, large access site with a cumulative diameter equivalent to the sum of all sheaths, the risk of bleeding complications is increased.

It is thus obvious that when the need for multiple access sites for a single vessel is present it is best to access the vessel from distinct entry points.

As such, a method, device, and system which would facilitate vascular access from separate sites after a first wire has been placed in the vessel is highly desirous and is the purpose of this invention.

### SUMMARY

In accordance with the preferred embodiments, a vascular access device comprises a bridge having first and second bores formed through bridge. A dilator is provided in the first bore and a needle guide is provided in the second bore. The second bore is formed at a non-zero angle with respect to the first bore such that the needle guide is at the same non-zero angle with respect to the dilator.

A needle and wire are inserted into a first blood vessel access site. The needle is then removed leaving just the wire in place. The vascular access device is then used. The dilator of the vascular access device is advanced over the wire and into the target blood vessel. A second needle is then inserted into the needle guide of the vascular access device. The angle between the needle guide and the dilator is such that the needle automatically is advanced through a second puncture site of the same blood vessel. Once the distal tip of the needle enters the blood vessel, a second wire is inserted through the needle and into the blood vessel. The vascular access device is removed leaving just the two wires in place and inserted into separate sites in the same blood vessel.

At least one embodiment of the invention is directed to a vascular access device that comprises a bridge that has first and second bores therethrough and a dilator in the first bore. The dilator extends from the bridge on opposing sides of the bridge. The vascular access device also comprises a needle guide in the second bore. The needle guide extends from at least one of the opposing sides. The second bore is at a non-zero angle with respect to the first bore which causes the needle guide to be at the same non-zero angle with respect to the dilator. The dilator extends a longer distance from one of said opposing sides than the needle guide.

### BRIEF DESCRIPTION OF THE DRAWINGS

For a detailed description of exemplary embodiments of the invention, reference will now be made to the accompanying drawings in which:

FIG. 1 shows a vascular access device in accordance with various embodiments of the invention;

FIG. 2 illustrates the non-zero angle at which a dilator and a needle are arranged in accordance with a preferred embodiment of the invention;

FIG. 3 depicts a needle that can inserted into the vascular access device;

FIG. 4 illustrates the vascular access device of FIG. 1 with a needle inserted therein in accordance with a preferred embodiment of the invention;

FIG. 5 shows a side view of the vascular access device of FIG. 4 in accordance with a preferred embodiment of the invention;

FIGS. 6-12 show a sequence of steps by which a health care provider uses the vascular access device to obtain a two-site entrance into a blood vessel in accordance with preferred embodiments of the invention; and

FIG. 13 shows an illustrative embodiment in which a dilator that is part of the vascular access device has an inflatable balloon at its distal end to facilitate the puncturing of the vessel at the second site.

### DETAILED DESCRIPTION

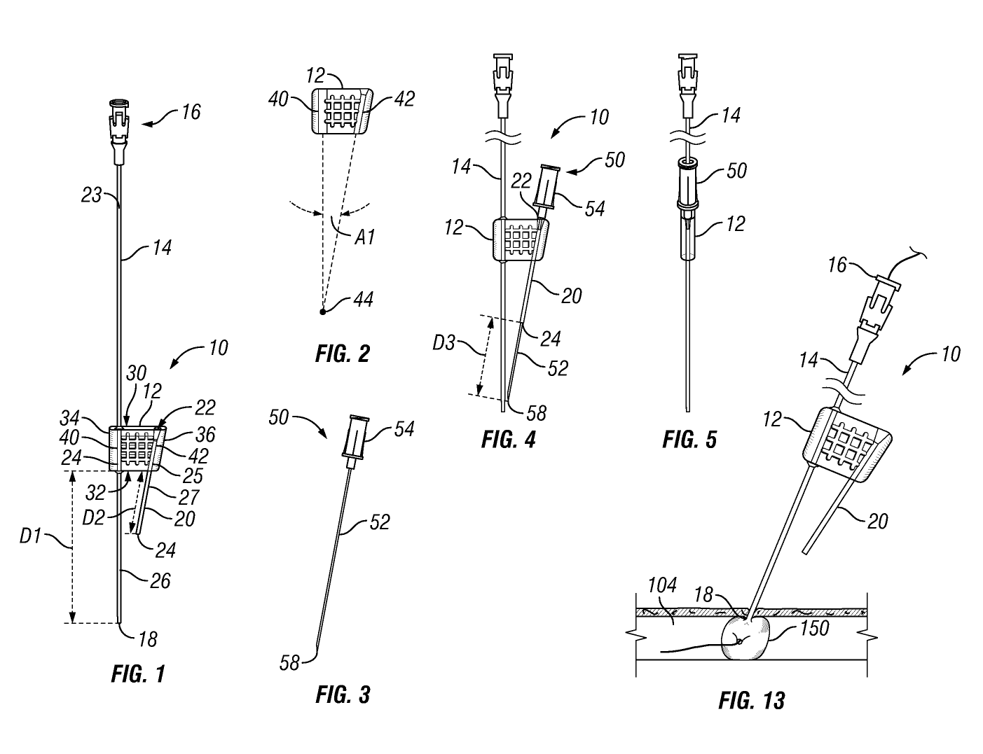

FIG. 1 illustrates a vascular access device 10 in accordance with a preferred embodiment of the invention. As shown, the vascular access device 10 comprises a bridge 12 and is useful to facilitate dual access to a blood vessel (vein, artery). The shape of the bridge 12 of the preferred embodiment is a quadrilateral and specifically a trapezoid as shown in FIG. 1. However, the shape of the bridge can be other than that shown in FIG. 1 (e.g., square, rectangular, etc.). The bridge 12 of FIG. 1 comprises four sides 30, 32, 34, and 36. Opposing sides 30 and 32 are parallel to each other and the other opposing sides 34 and 36 are not parallel to each other.

The bridge 12 may be made from plastic or other suitable material and may be a sold block of material if desired, other than the bores 40 and 42 explained below. The bridge 12 comprises a first bore 40 and a second bore 42 extending between opposing sides 30 and 32. The second bore 42 preferably is at a non-zero angle with respect to the first bore, but can be at a zero angle (i.e., parallel) as well, if desired. Referring briefly to FIG. 2, the angle between the two bores 40, 42 is designated as A1. In accordance with preferred embodiments of the invention in which the angle is non-zero, the angle A1 is in the range of 11 degrees to 35 degrees. The angle A1 depends on the patient and the application and thus may vary as desired. In at least one embodiment, the angle A1 is 11.6 degrees. Different size bridges 12 or bridges with different angles A1 may be provided to accommodate the varying needs of the health care provider attempting to achieve dual access to a common blood vessel.

Referring again to FIG. 1, the first bore 40 accepts at least a portion of a dilator 14. The dilator 14 in the preferred embodiment of FIG. 1 has a portion 23 that extends from side 30 of bridge 12, a portion 24 that extends through bore 40, and another portion 26 that extends from opposing side 32 of the bridge 12. The dilator 14 preferably is one piece but can be multiple pieces if desired. The dilator 14 comprises a proximal end 16 and a distal end 18. The distal end 18 of the dilator is the end that is inserted through the patient's skin and into the target blood vessel. The dilator 14 preferably is adhered to bore 40 by, for example, an adhesive. In such embodiments, the dilator 14 is permanently affixed to (not removable from) the bridge 12. That is, during its intended usage, the dilator 14 does not separate from the bridge 12. In other embodiments, the dilator is removable. In yet other embodiments, the dilator 14 can be locked in place in the bridge at any of a plurality of positions (i.e., repositionable). In yet another embodiment, the dilator is rotatable within the bridge. The second bore 42 of bridge 12 accepts at least a portion of a needle guide 20. The needle guide 20 has a portion that 25 that extends through the bore 42 and another portion 27 that extends from side 32 of the bridge 12. The needle guide 20 comprises a proximal end 22 into which a needle can be inserted (pointed end in first) and a distal end 24 from which the pointed end of the needle exits. The needle guide 20 preferably is adhered to bore 42 by, for example, an adhesive. In such embodiments, the needle guide 20 is permanently affixed to the bridge 12. That is, during its intended usage, the needle guide 20 does not separate from the bridge 12.

Because the bores 40 and 42 are, in some embodiments, at a non-zero angle with respect to each other and because dilator 14 and needle guide 20 reside within the bores, dilator 14 and needle guide 20 also are arranged at the same angle A1 as their respective bores.

The portion 26 of the dilator 14 that extends from side 32 of the bridge 12 extends a distance D1 from side 32. The portion 27 of needle guide 20 that extends from side 32 of the bridge 12 extends a distance D2 from side 32. In accordance with the preferred embodiments, D1 is longer than D2 as depicted in FIG. 1. The dimensions D1 and D2 will depend on the patient and the application and thus may vary as desired. Different size vascular access devices 10 or vascular access devices 10 with different lengths for D1 and/or D2 may be provided to accommodate the varying needs of the health care provider attempting to achieve dual access to a common blood vessel.

FIG. 3 shows an illustrative embodiment of a needle 50 that can be inserted into needle guide 20 of the vascular access device 10. The needle comprises a pointed shank 52 with a head 54 at the shank's proximal end. The shank 52 comprises a pointed distal end 58. The gauge of the needle depends on the application and patient for its intended use.

FIG. 4 illustrates the vascular access device 10 with the needle 52 inserted into the needle guide 20. The length of the needle 52 is such that, when head 54 touches the bridge 12 or the proximal end 22 of the needle guide 20, the pointed, distal end 58 of the needle 50 extends a distance D3 past the distal end 24 of the needle guide. When fully inserted, the pointed distal end 58 of the needle approaches but does not extend as far as the distal end 18 of the dilator 14.

In some embodiments, the bridge 12 lacks a needle guide. In such embodiments, the needle 50 is integrated into (i.e., permanently affixed to) the bridge and thus not removable.

FIG. 5 shows a side view of the vascular access device 10 of FIG. 4 with needle 50 inserted therein.

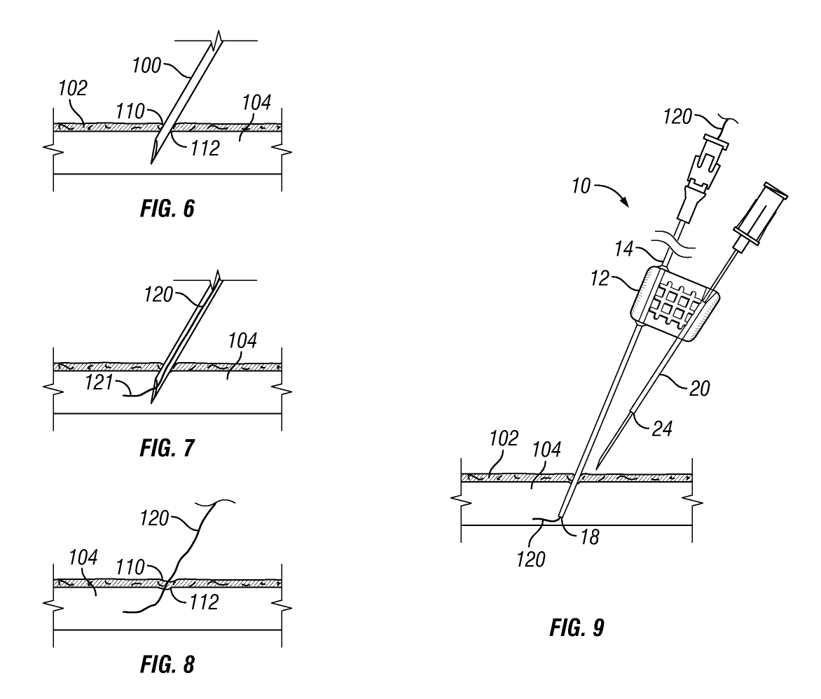

FIGS. 6-12 show a sequence of events using the vascular access device 20 of the preferred embodiments. In FIG. 6, a health care provider inserts a needle 100 through the patient's skin 104 and into the target blood vessel 104. The target blood vessel may be an artery or a vein. In inserting the needle 100, a puncture site 110 through the skin 102 and a puncture site 112 through the vessel 104 are created.

In FIG. 7, the health care provider inserts a wire 120 through the inserted needle 100. The distal end 121 of the wire extends into the interior of the target blood vessel 104. In FIG. 8, the needle 100 is withdrawn by the health care provider leaving just the wire 120 in place and inserted into the target blood vessel 104.

In FIG. 9, the health care provider advances the dilator 14 of the vascular access device 10 over the wire 120. The distal end 18 of the dilator 14 is advanced into the target blood vessel 104 guided by wire 120. As the vascular access device 10 is advanced, the needle guide 20 is also advanced towards the patient's skin 102. When the dilator 14 is fully advanced so that the distal end 18 of dilator 14 is in the target blood vessel, the distal end 24 of the needle guide 20 is near the patient's skin 104.

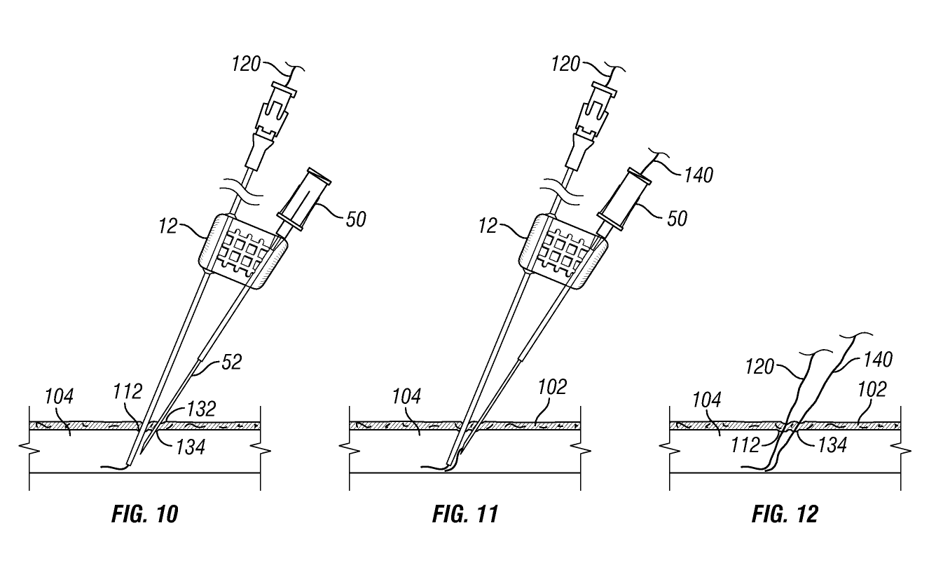

In FIG. 10, the health care provider advances a needle 50 into the needle guide 20 of the vascular access device 10. The angle (A1) of the needle guide 20 is such that the needle 50, when advanced, inherently inserts through the skin (at site 132) and into the same blood vessel 104 at a separate site 134. The separate site 134 is spaced apart from site 112 of guide wire 120 at a desired distance so as to mitigate the problems noted previously.

In FIG. 11, a second wire 140 is inserted into needle 50 and into blood vessel 104. In FIG. 12, the vascular access device 10 (bridge 12, dilator 14, needle 50, and needle guide 20) is removed thereby leaving only wires 120 and 140 both inserted through separate sites into the same blood vessel.

FIG. 13 illustrates another embodiment in which the distal end 18 of the dilator 14 has an inflatable balloon 150. The balloon is inflated via injection of air or liquid into the proximal end 16 of the dilator 14. Inflation of the balloon will decrease flow in the vessel, increasing the intravascular pressures and dimensions of the vessel. This will facilitate the penetration of the needle 50 through needle guide 20 into the vessel 104.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

What is claimed is:

1. A vascular access device, comprising:
a bridge having first and second bores therethrough;
a dilator in the first bore;
a needle guide in the second bore;
wherein the second bore is at an angle with respect to the first bore such that the needle guide is at said angle with respect to the dilator.

2. The vascular access device of claim 1 wherein the angle is in the range of 11 degrees to 35 degrees.

3. The vascular access device of claim 1 wherein the angle is zero degrees.

4. The vascular access device of claim 1 further comprising a first wire running through the dilator.

5. The vascular access device of claim 1 further comprising a needle running through the needle guide.

6. The vascular access device of claim 1 further comprising an inflatable vascular balloon at a distal end of the dilator.

7. The vascular access device of claim 1 wherein a portion of the dilator, containing a distal end, extends from the bridge a first distance and a portion of the needle guide, containing a distal end, also extends from the bridge a second distance, and wherein the second distance is shorter than the first distance such that the distal end of the dilator enters a blood vessel of a patient before a needle contained in the needle guide enters the same blood vessel.

8. The vascular access device of claim 1 wherein the dilator is not removable from the bridge.

9. The vascular access device of claim 1 wherein the dilator is removable from the bridge.

10. The vascular access device of claim 1 wherein the dilator can be locked in place in the bridge at any of a plurality of positions.

11. The vascular access device of claim 1 wherein the dilator is rotatable within the bridge.

12. A vascular access device, comprising:
a bridge having first and second bores therethrough;
a dilator in the first bore, said dilator extends from said bridge on opposing sides of said bridge;
a needle or needle guide in the second bore, said needle or needle guide extends from at least one of said opposing sides;
wherein the second bore is at an angle with respect to the first bore such that the needle or needle guide is at said angle with respect to the dilator; and
wherein said dilator extends a longer distance from one of said opposing sides than the needle or needle guide.

13. The vascular access device of claim 12 wherein the angle is in the range of 11 degrees to 35 degrees.

14. The vascular access device of claim 12 further comprising a wire extending through each of the first and second bores into a blood vessel.

15. The vascular access device of claim 14 wherein the wires remain in place inserted into the blood vessel after the bridge with dilator and needle or needle guide is removed.

16. The vascular access device of claim 14 wherein the wires extend into the blood vessel at two different sites.

17. The vascular access device of claim 14 wherein the dilator is selected from a group consisting of a dilator that is fixed in place in the bridge, removable from said bridge, rotatable within the bridge, and repositionable within the bridge.

18. A method, comprising:
inserting a first wire into a blood vessel;
advancing a distal end of a dilator over the first wire into the blood vessel, said dilator being mounted in a vascular access device having a needle guide formed at an angle respect to the dilator; and
with the distal end of the dilator in said blood vessel, advancing a needle into said needle guide until the needle penetrates the same blood vessel at a different site than the distal end of the dilator.

19. The method of claim 18 further comprising advancing a distal end of a second wire through said needle and into said blood vessel at said different site.

20. The method of claim 19 further comprising removing said dilator vascular access device, dilator and needle leaving said first and second wires in place in said blood vessel.
